(19) United States
(12) Patent Application Publication
(10) Pub. No.: US 2024/0344091 A1
(43) Pub. Date: Oct. 17, 2024

(54) PROCESS AND PLANT

(71) Applicant: Cemvita Factory, Inc., Houston, TX (US)

(72) Inventors: Zachary Richard BROUSSARD, Houston, TX (US); TJ TIDWELL, Houston, TX (US); Michael SAMUEL, Houston, TX (US); Edward John KOCH, Houston, TX (US); Raja Subramanian RAMANATHAN, Houston, TX (US); Renata Amanda GONÇALVES, Houston, TX (US); Aaron TREVINO, Houston, TX (US); Addien WRAY, Houston, TX (US); Bárbara de Freitas MAGALHÃES, Houston, TX (US); Edris TAHER, Houston, TX (US)

(21) Appl. No.: 18/619,150

(22) Filed: Mar. 27, 2024

Related U.S. Application Data
(60) Provisional application No. 63/492,603, filed on Mar. 28, 2023.

(30) Foreign Application Priority Data
Apr. 18, 2023 (GB) 2305691.4

(51) Int. Cl. C12P 3/00; C12M 1/00

(52) U.S. Cl. CPC C12P 3/00; C12M 23/52; C12M 41/14

(57) ABSTRACT

The present application provides a process and plant for the microbial production of hydrogen from the site of hydrocarbonaceous deposit, the process comprising modifying the composition of the deposit through the introduction into or into the vicinity of the deposit of at least one hydrogen producing microorganism, wherein the at least one hydrogen producing microorganism is provided on site by means of a transportable microbiological incubator, the plant comprising means in the form of a transportable microbiological incubator for generating at least one hydrogen producing microorganism; and means for supplying the at least one at least one hydrogen producing microorganism into the hydrocarbonaceous deposit.

This application claims priority to U.S. Provisional Application No. 63/492,603, filed Mar. 28, 2023 and to GB Application No. 2305691.4, filed Apr. 18, 2023. The entirety of the aforementioned applications are incorporated herein by reference.

### FIELD

The present application concerns a process and plant for the microbiological production of hydrogen from a hydrocarbonaceous deposit, especially a subterranean deposit, employing a transportable microbiological incubator for the purposes of delivering one or more hydrogen-producing microorganisms into or into the vicinity of the hydrocarbonaceous deposit.

### BACKGROUND

Hydrogen is an important fuel and chemical process substrate. It is known in the art to use microbes to produce hydrogen from hydrocarbon substrates.

WO2005115648 describes a process for characterizing and then manipulating the environment of fermentative syntrophic microorganisms naturally present in a petroleum-bearing subterranean formation in order to promote microbial generation of hydrogen in the formation.

WO2015052806 similarly describes the use of an Fe (III) activator compound to stimulate subterranean microbial hydrogen and methane and also suggests ex situ cultivation and subsequent re-injection of microbes naturally occurring in the subterranean environment.

WO2005113784 describes a method for enhancing microbial production of hydrogen from a hydrocarbon rich deposit. The disclosure favors achieving this by stimulating the metabolic activities of indigenous microorganisms within the deposit, including by the introduction of exogenous (possibly genetically modified) organisms having metabolic capabilities of interest. These metabolic capabilities are not defined except insofar as their impact is to improve net hydrogen production, and contextually this seems to mean by inhibiting the consumption of hydrogen rather than by metabolization of hydrocarbons to hydrogen within the deposit. This document therefore fails to appreciate or to disclose the introduction into the deposit of further microorganisms which are non-native to the deposit and which themselves are capable of metabolizing hydrocarbons to molecular hydrogen and which serve to increase hydrogen production in the deposit by positively diversifying the microbiological abundance of microorganisms in the deposit.

WO0234931 describes a method of generating and recovering methane from solid carbonaceous deposits. This disclosure suggests to inject bacterial consortia into such deposits and recognizes that hydrogen as well as methane may be produced, but methane production is the clear objective of the disclosure and fermentative hydrogen producers are envisioned as being useful only insofar as they provide a feedstock for methanogenesis.

Our co-pending application PCTUS2022076925, the contents of which are hereby incorporated by reference, describes a process for the microbiological production of hydrogen from a hydrocarbon-rich deposit. The process comprises modifying the composition of the deposit by the introduction of hydrogen producing microorganisms selected to diversify the microbiological abundance of hydrogen-producing microorganisms in the deposit and for the preferential production of hydrogen over methane.

Singh et al., “Overview of Carbon Capture Technology: Microalgal Biorefinery Concept and State-of-the-Art”, Frontiers in Marine Science, 6, 2019, details an overview of carbon capture technology, and in particular microalgal biorefineries, as a means to combat climate change. Singh et al. detail how microalgae can be used to convert raw materials into high and low value products and fuels derived from biomass.

Barnhart et al., “Enhanced coal-dependent methanogenesis coupled with algal biofuels: Potential water recycle and carbon capture”, International Journal of Coal Geology, 171, 2017, 69-75, details methods for stimulating the production of methane from coal bed methanogenesis by introducing further additives to the coal bed to stimulate the activity of the native microorganisms.

Davis', “Organic amendments for enhancing microbial coalbed methane production”, Montana State University, 2017, details the use of organic amendments, i.e. the addition of microbes and/or additives, to enhance the microbial processes for coal-to-methane produced coalbed methane, a form of natural gas found in subsurface coal beds wherein the methane is generated by native microbes to the coal bed. The process detailed therefore focuses on the addition of additives to enhance an already natural process.

Veshareh et al “The light in the dark: In-situ biorefinement of crude oil to hydrogen using typical oil reservoir Thermotoga strains”, International Journal of Hydrogen Energy, Volume 47 (8), 2022 (https://doi.org/10.1016/j.ijhydene.2021.11.118), details the use of bacteria extracted from hydrocarbon reservoirs with the use of native bacteria such as Thermotoga strains, to convert hydrocarbons into hydrogen. This paper details a range of different challenges faced in translating the study from the lab to field applications and therefore does not provided a suitable disclosure of the use of a transportable microbiological incubator.

US2009/0029879 details a process for enhancing oil recovery from an oil well. The document describes providing microbes to an oil well from a transportable field laboratory assembly comprising a laboratory unit comprising at least one anaerobic bioreactor. However, the process disclosed in this document is not for use onsite or for the mobile production of microorganisms. There is no consideration provided to the production of hydrogen or towards the disclose of the introduction into the deposit of further microorganisms which are non-native to the deposit and which themselves are capable of metabolizing hydrocarbons to molecular hydrogen and which serve to increase hydrogen production in the deposit by positively diversifying the microbiological abundance of microorganisms in the deposit.

In the exemplified prior art examples, the primary focus concerns the manipulation of indigenous microbial populations or their environment, in some cases with the aid of other microbes which inhibit hydrogen consumption or which are themselves methanogenic. Little attention has been paid to the practical difficulties of providing on-site microbiological reagents, especially in circumstances where the site of the hydrocarbon deposit is remote, inaccessible or otherwise inconvenient for the establishment of fixed plant.

### SUMMARY

According to a first aspect of the present application there is provided a process for the microbial production of hydrogen from the site of hydrocarbonaceous deposit, the process comprising modifying the composition of the deposit through the introduction into or into the vicinity of the deposit of at least one hydrogen producing microorganism, wherein the at least one hydrogen producing microorganism is provided on site by means of a transportable microbiological incubator.

The at least one hydrogen producing microorganism may be a non-native microorganism. By “non-native” is meant that the microorganism does not occur naturally at the site, or does not occur naturally in abundance at the site. An “abundance” may be defined as comprising, in a sample taken from the site, over 10%, over 5% or over 1% w/w of all microorganisms in the sample.

The term “into the vicinity” is to be construed as the at least one hydrogen producing microorganism being introduced in fluid communication with the hydrocarbonaceous deposit.

The incubator is preferably “transportable” in the sense that it can be shipped essentially operationally intact from one location to another. Preferably the incubator is containerized in operationally intact form. A suitable form of containerization may be provided in the form of a container substantially similar in size and shape to a standard shipping container. By “operationally intact” is meant that the incubator may be commissioned at a first location such that at least one hydrogen producing microorganism is nurtured within the incubator and subsequently transported to a second location (optionally the site of the deposit), whereat the at least one hydrogen producing microorganism continues to be nurtured within the incubator.

The incubator may be provided with means for culturing microbiological materials, including means for providing microbiological materials in the incubator with one or more nutrients and/or adjuvants for encouraging or selectively encouraging the growth of the at least one hydrogen producing microorganism in the incubator, and with means for maintaining conditions of temperature, pressure and/or chemical environment (e.g. salinity) within the incubator conducive to the incubation of the microorganism.

The incubator may therefore be provided in the form of a containerized and transportable unit which may be shipped to site and provided with means for supplying the at least one hydrogen producing microorganism from the incubator into or into the vicinity of the hydrocarbonaceous deposit.

The hydrogen producing microorganism contained in the transportable microbiological incubator may be selected positively to diversify the microbial abundance of hydrogen-producing microorganisms in the deposit when charged thereto, and optionally also for the preferential production of hydrogen over methane. This will especially be the case when the hydrogen producing microorganism is a non-native hydrogen producing microorganism.

The process may be used in liquid hydrocarbonaceous reservoirs. The liquid hydrocarbonaceous reservoirs may comprise at least one well for injection and at least one well for production.

According to a second aspect of the present application there is provided plant for the microbial production of hydrogen from a hydrocarbonaceous deposit comprising: means in the form of a transportable microbiological incubator for generating at least one hydrogen producing microorganism; and means for supplying the at least one at least one hydrogen producing microorganism into the hydrocarbonaceous deposit.

The aforementioned plant may additionally comprise means for extracting from the hydrocarbonaceous deposit a product stream comprising at least hydrogen gas generated by the microbiological action in the deposit of the at least one hydrogen producing microorganism.

The product stream may comprise useful product streams besides hydrogen—such as oil and/or methane. The product stream may further comprise an aqueous phase deriving at least in part from an aqueous carrier for the at least one hydrogen producing microorganism supplied to the hydrocarbonaceous deposit.

Product stream phases may be separated and any aqueous phase recycled to the deposit, optionally with the incorporation of fresh microbiological material from the incubator.

A three phase separator may be used to isolate independently the gaseous, oleaginous and aqueous streams from the product stream.

The plant provides a versatile means of converting hydrocarbon deposits in situ. Once a target well or other site containing such hydrocarbon deposits has been identified, its geological and geochemical characteristics may be assessed, e.g. by core flood methodology which is the subject of our co-filed application P13143US. In (for example) this manner a suitable microbiological agent (or a consortia of microbial agents) may be identified for the most efficacious extraction of hydrogen from that particular site. The transportable microbiological incubator may be charged with the appropriate microbiological agent(s) and any suitable nutrients and/or adjuvants, maintained in the incubator under conditions effective for functional maintenance of the microbiological agent(s), and transported in such condition to the site whereupon it can be established by means of suitable connective infrastructure as a “plug and play” unit feeding the microbes and associated materials directly into or into the vicinity of the hydrocarbon deposits. Generally an aqueous carrier may be used to transport the microbiological agent(s) into the (vicinity of the) deposit, and such aqueous carrier may be provided at least in part by an aqueous recycle stream from the site.

The present application provides several clear advantages over the existing state of the art. Through integration of a transportable microbiological incubator with a process converting a hydrocarbonaceous deposit to hydrogen using microorganisms, a more efficient, versatile and technically improved conversion of hydrocarbon deposits to hydrogen can be achieved.

For the avoidance of doubt, all features relating to the method of the present application also relate, where appropriate, to the plant of the present application and vice versa.

### BRIEF DESCRIPTION OF THE DRAWINGS

The invention will now be more particularly described with reference to the following Examples and Figure, in which;

FIG. 1 is a schematic flow diagram of a process operating in accordance with one aspect of the invention.

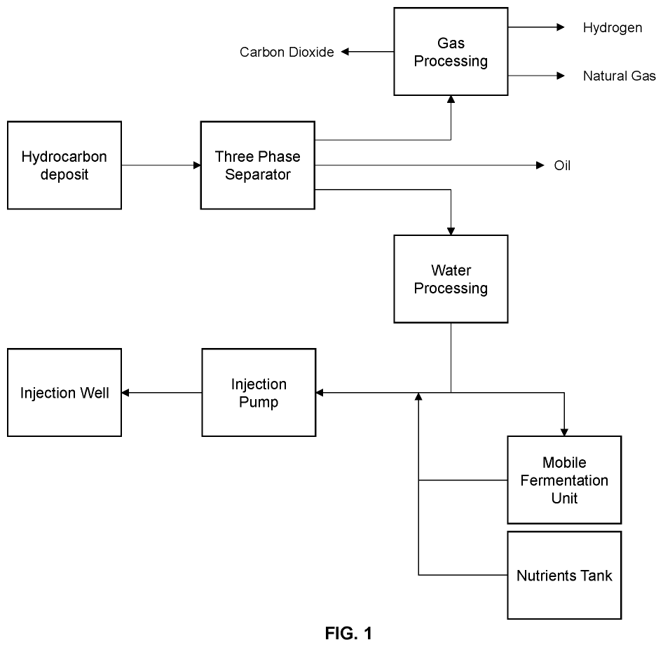

The plant, as illustrated by FIG. 1, comprises a hydrocarbonaceous deposit, a three phase separator, a gas processing station, an injection well, an injection pump, a transportable microbiological incubator, and a nutrient tank.

The hydrocarbon rich deposit may contain crude oil, natural gas or coal. The deposit may comprise a porous rock formation.

The three phase separator separates water, oil and gas components collected from the hydrogen-rich deposits. The gaseous components are collected and are provided to a gas processing station. The liquid hydrocarbon components are collected and may be subject to further processing, or sold as an end product. The aqueous components are collected and may be provided to a water processing unit, or disposed of.

The gas processing station is used to process the gaseous products collected from a deposit, and may be used to separate hydrogen, natural gas, and carbon dioxide. In some embodiments of the present application, the carbon dioxide may be collected usefully re-processed. The gas processing station also provides a means for cleaning the product gases for further use, or for sale as an end product.

The water processing unit may be responsible for cleaning the water collected by the three phase separator. In some embodiments of the present application, undesirable volatile fatty acids (VFAs) may be removed in the water processing unit. The water collected by the three phase separator may further be used as a supply to the injection pump. An advantage of recycling the water from the extraction stages is that waste water is reduced.

The transportable microbiological incubator contains the at least one hydrogen producing microorganism capable of modifying the composition of the hydrogen-rich deposit. The transportable microbiological incubator may comprise at least one bioincubator responsible for producing the hydrogen producing microorganisms. In some embodiments, the transportable microbiological incubator may be supplied with water from the water processing unit.

The nutrient tank may contain the nutrients or adjuvants required by the hydrogen producing microorganisms. In preferred embodiments of the present application, the transportable microbiological incubator and the nutrient tank may be contained in the same container, for example a shipping container.

The flow of microbes, nutrients from the transportable microbiological incubator and water passes to an injection pump, which injects the reaction mixture into an injection well, which is the deposit wherein the microbial production of hydrogen from a deposit occurs.

In a further embodiment according to the second aspect of the present application, the plant may comprise a core flood analysis plant for determining the microbe (or consortia of microbes) required for the most efficient conversion of the deposit into hydrogen. Any such or alternative analysis plant may be provided off-site—optionally at a site of location of the microbiological incubator prior to its transportation to the site of the deposit.

For the avoidance of doubt, all features relating to the method of the present application also relate, where appropriate, to the plant of the present application and vice versa.

It should be apparent that any of the embodiments of the invention, and each or any of their described variants, may be provided in combination with the first or second aspects of the invention and each or any of its described variants and/or in combination with any one or more of each other.

### DETAILED DESCRIPTION

In one embodiment the transportable microbiological incubator is a containerized unit, comprising a means of growing microorganisms, such as a bioincubator. Such bioincubators may be similar in their construction to commercially available fermentation units used in industrial processes such as brewing. The bioincubators may be charged with the at least one hydrogen producing microorganism, and/or a consortia of microbes and/or microorganisms.

In some embodiments of the present application, the transportable microbiological incubator may comprise at least one bioincubator, preferably more than one bioincubator. In a preferred embodiment, the bioincubators will be situated inside a mobile container, for example a modified shipping container, which can be transported between locations easily.

The bioincubators contained within the transportable microbiological incubator may comprise a stirrer, a thermal jacket, a cooling means, pH sensor and/or a thermometer.

In some embodiments, the bioincubators contained within the transportable microbiological incubator may be selectively controlled by a control plant, which can be used to monitor the temperature, pH, feedstock addition, and the characteristics of the microbiological population(s) contained within.

The transportable microbiological incubator may be charged with microorganisms identified through core flood methodology. Core flood methodology involves determining the response of the geological material of a hydrocarbon deposit to treatment with different microorganisms and nutrient conditions with regards to the efficacy of hydrogen generation from the material is assessed. In a core flood exploration a geological sample from the site is maintained under conditions of pressure, temperature and/or chemical environment intended to be replicative of the in situ conditions. The response of the sample to different microbial populations and nutrient conditions may therefore be assessed and the most suitable reagents determined. In preferred embodiments of the present application, once such determination has been made the preferred microbial reagents and associated nutrient/adjuvant package can be charged to a transportable microbiological incubator and transported to site.

In some embodiments of the present application the transportable microbiological incubator may be accompanied by—or have incorporated within it—a nutrient tank. The nutrient tank may contain any required nutrients or adjuvants for the growth of the hydrogen producing microorganism(s). In a preferred embodiment of the present application, the nutrient tank is also contained within the transportable microbiological incubator, providing a single compact unit that is easier to transport to and from hydrocarbon deposit sites.

The hydrogen producing microorganism may be:
a. a microorganism not naturally present in the hydrocarbonaceous deposit; and/or
b. of a strain of microorganisms not naturally present in the hydrocarbonaceous deposit; and/or
c. of a species of microorganisms not naturally present in the hydrocarbonaceous deposit; and/or
d. of a genus of microorganisms not naturally present in the hydrocarbonaceous deposit;
e. a microorganism naturally present in the hydrocarbonaceous deposit; and/or
f. a microorganism naturally present in the hydrocarbonaceous deposit but genetically modified to increase (relative to the naturally present microorganism) its propensity for hydrogen production by the metabolization by that microorganism of one or more hydrocarbons contained within the deposit.

The at least one hydrogen producing microorganism may be one of a plurality of different hydrogen producing microorganisms, strains of microorganisms, species of microorganisms, genera of microorganisms and/or naturally occurring but genetically modified organisms introduced into the deposit. Genetic manipulation of microorganisms naturally present in the deposit to form non-native species may be effected by directed evolution or other form of synthetic biology. The plurality may be greater than two, greater than three, greater then four, greater than five and/or greater than ten.

The hydrogen producing microorganism(s) may have a propensity to metabolize one or more hydrocarbons contained within the deposit to molecular hydrogen (preferably in preference to methane) such that the yield of production of molecular hydrogen (H₂) from the metabolization is higher than the yield of production of methane by at least 1%, by at least 10%, by at least 100% and/or by at least 1000%.

The hydrogen producing microorganism(s) may be introduced into the deposit from the transportable microbiological incubator and accompanied during, after or upon its introduction by at least one nutrient selected to promote the growth of said microorganism and introduced into the deposit for that purpose, optionally wherein the nutrients are supplied from a nutrient reservoir, optionally a nutrient reservoir contained within the incubator.

Conditions within the incubator may at least to some extent replicate one or more conditions (of temperature, pressure and/or chemical environment) of the site of hydrocarbonaceous deposit, and the hydrogen producing microorganism(s) may therefore be isolated from the incubator before introduction into the deposit in a condition adapted for optimized hydrogen-producing efficacy upon their introduction.

The at least one nutrient may be selected preferentially to promote the growth of the said microorganism in preference to at least one, to at least some or to all of any native microorganisms in the deposit.

The nutrient may comprise one or more of:
g. one or more salts selected from:
   i. phosphates; and/or
   ii. halides; and/or
   iii. nitrates, ammonium salts, nitrogenous salts; and/or
h. one or more carbohydrates selected from:
   i. sugars; and/or
   ii. starches; and/or
i. one or more vitamins; and/or
j. complex nutrients, optionally comprising yeast extracts; corn steep liquor; biomass; bacterial an/or algal biomass.

As will be apparent from Example 1 below it is particularly advantageous to include at least one carbohydrate and/or complex nutrient in the at least one nutrient.

The hydrogen producing microorganism may be introduced into the deposit and accompanied during, after or upon its introduction by at least one pH regulator selected to regulate the pH environment in which the microorganism resides in the deposit and introduced into the deposit for that purpose. The pH regulator may be selected to regulate the pH of the hydrogen producing microorganism environment in the deposit to a pH within the range of from about 5 to about 9, from about 6 to about 8 and/or from about 6 to about 7.

The pH regulator may optionally also serve as a nutrient—for example, phosphate can acts as both a nutrient and as a buffering agent.

The hydrogen producing microorganism may be introduced into the deposit and accompanied during, after or upon its introduction by at least reducing agent which may or may not be included as part of the nutrient package. Suitable reducing agents include thioglycolic acid (and salts such as sodium thioglycolate), cysteine HCl, Na2S, FeS, dithiothreitol, sodium dithionite, ascorbic acid, oxalic acid, sodium sulfite, sodium metabisulfite, 2-mercaptoethanol, sodium pyruvate, glutathione and compatible mixtures of two or more thereof.

The hydrocarbonaceous deposit is preferably a liquid hydrocarbonaceous deposit, e.g oil/bitumen/heavy oil.

The at least one hydrogen producing microorganism may have a genus of Syntrophobacter, Syntrophus, Syntrophomonas, Thermoanaerobacter, Thermotoga, Pseudothermotoga, Thermoanaerobacterium, Fervidobacterium, Thermosipho, Haloanaerobium, Acetoanaerobium, Anaerobaculum, Geotoga, Petrotoga, Thermococcus, Pyrococcus, Clostridium, Enterobacter, Klebsiella, Ethanoligenens, Pantoea, Escherichia, Bacillus, Caldicellulosiruptor, Pelobacter, Caldanaerobacter, Marinitoga, Oceanotoga, Defluviitoga, Kosmotoga, Caloranaerobacter or a combination or mixture thereof.

The hydrogen producing microorganism may express at least one protein selected from hydrogenases, dehydrogenases, hydroxylases, carboxylases, esterases, hydratases and acetyltransferases having an amino acid sequence at least 95% identical to a sequence expressed by an upregulated or downregulated gene selected from mth (EC 1.12.98.2), mrt, hycA (ID: 45797123), fdhF (ID: 66346687), fhlA (ID: 947181), ldhA (ID: 946315), nuoB (ID: 65303631), hybO (ID: 945902), fdh1, narP, ppk or Pepc by expressing a non-native protein expressing nucleotide sequence, wherein an amount of hydrogen produced or protein produced by the hydrogen producing microorganism is greater than that produced relative to a control microorganism lacking the non-native protein expressing nucleotide sequence.

The hydrogen producing microorganism may be a recombinant microorganism.

The recombinant microorganism may express at least one Coenzyme M reductase and or dehydrogenase protein having a gene sequences at least 95% identical to SEQ ID NO. [mmg:MTBMA_c15480], [mth:MTH_1015], [mmg:MTBMA_c15520], [mmg:MTBMA_c15490], [mth:MTH_1166], [mth MTH_1167], [eco:b4346], [eco:b4345], [ag:AAA22593], [mea:Mex_1p4538, [mea:Mex_1p4535], [ag:ACS29499], [ag:CAH55641], [mrd:Mrad2831_0508], by expressing a non-native Coenzyme M reductase and or dehydrogenase expressing nucleotide sequence.

Preferably, an amount of hydrogen produced or protein produced by the hydrogen producing microorganism is greater than that produced relative to a control microorganism lacking the non-native protein expressing nucleotide sequence.

The environment of the hydrocarbonaceous deposit and the introduced hydrogen producing microorganism may constitute an enclosed bioreactor, being a bioreactor subterranean formation, a bioreactor landfill enclosure, or a combination thereof.

In this case there is provided in accordance with the aforesaid first aspect of the invention and any or each of its described variants a method of increasing hydrogen production from an enclosed bioreactor (as constituted by the environment of the hydrocarbonaceous deposit and the introduced hydrogen producing microorganism) comprising: providing a baseline reaction mixture in the enclosed bioreactor, wherein the baseline reaction mixture includes a hydrocarbon, water, and a baseline amount of at least one microorganism; producing baseline microorganism data on an identity and a baseline percentage of the at least one microorganism, relative to a baseline total percentage of microorganisms in the baseline reaction mixture, by performing DNA and/or RNA sequencing of a baseline microorganism sample from the baseline reaction mixture; measuring a baseline amount of hydrogen in a baseline gas sample of gasses collected from the enclosed bioreactor; increasing hydrogen production from the enclosed bioreactor by forming a synthetic reaction mixture including at least one hydrogen producing microorganism supplied from a transportable microbiological incubator, and harvesting the hydrogen from the enclosed bioreactor at a hydrogen harvesting rate by separating the hydrogen from other gasses and transferring the hydrogen into a hydrogen storage container.

The synthetic reaction mixture is formed by: adding at least one hydrogen producing microorganism from the transportable microbiological incubator until a percentage of the hydrogen producing microorganism in the synthetic reaction mixture is at least 20% of a total amount of microorganisms in the synthetic reaction mixture.

The method may further comprise after providing the baseline reaction mixture, but before forming the synthetic reaction mixture, producing baseline environmental data from the baseline reaction mixture. The baseline environmental data may include one or more of the following measurements of a baseline environmental sample from the baseline reaction mixture: pH; temperature; water analysis; oxidation-reduction potential; pressure; dissolved oxygen; hydrocarbon concentrations; volatile fatty acids concentrations; cation concentration; anion concentration; concentration of gases (such as one or more of NH₃, CO₂, CO, H₂, H₂S and CH₄); salt concentration; and metal concentration. Core flood methodology may be used to investigate any one or more of these parameters.

The baseline microorganism sample and the baseline environmental sample may be the same or different.

The hydrogen harvesting rate may be at least about 0.1 L/hr, or at least about 1 L/hr, or at least about 10 L/hr, or at least about 100 L/hr. The hydrogen harvesting rate may be up to about 10⁶ L/hr, or up to about 10⁵ L/hr, or up to about 10⁴ L/hr, or up to about 10³ L/hr. The hydrogen harvesting rate may be from about 0.1 L/hr to about 10⁶ L/hr, or from about 0.1 L/hr to about 10³ L/hr, or from about 10³ L/hr to about 10⁶ L/hr.

The subterranean formation may include a natural formation, non-natural formation, a hydrocarbon-bearing formation, a natural gas-bearing formation, a methane-bearing formation, a depleted hydrocarbon formation, a depleted natural gas-bearing formation, a wellbore, or a combination thereof.

The bioreactor landfill enclosure may include a landfill that is enclosed by a building material. The building material may include at least one of a brick, a cement, a plastic, a non-natural rubber, a geomembrane of any kind, concrete, steel, a glass, or a combination thereof.

The hydrogen producing microorganism may be supplied to the deposit in combination with a hydrogen production enhancer, for example a biocidal inhibitor, a methanogenesis inhibitor, a sulfate reduction inhibitor, a nitrate reduction inhibitor, an iron reduction inhibitor, or any suitable combination thereof.

The biocidal inhibitor may be glutaraldehyde, a quaternary ammonium compound, formaldehyde, a formaldehyde releaser such as 3,3′-methylenebis[5-methyloxazolidine], dibromonitrilopropionamide, tetrakis hydroxymethyl phosphonium sulfate, chlorine dioxide, peracetic acid, tributyl tetradecyl phosphonium chloride, methylisothiazolinone, chloromethylisothiazolinone, sodium hypochlorite, dazomet, dimethyloxazolidine, trimethyloxazolidine, N-bromosuccinimide, bronopol, or 2-propenal, or a mixture thereof.

The methanogenesis inhibitor may be bromethane sulfonic acid, an aminobenzoic acid, 2-bromoethanesulfonate, 2-chloroethanesulfonate, 2-mercaptoethanesulfonate, lumazine, a fluoroacetate, nitroethane, or 2-nitropropanol, or a mixture thereof.

The sulfate reduction inhibitor may be a molybdate salt, a nitrate salt, a nitrite salt, a chlorate salt, or a perchlorate salt or a mixture thereof.

The nitrate reduction inhibitor may be sodium chlorate, a chlorate salt, or a perchlorate salt, or a mixture thereof.

The method of the invention may further comprise producing carbon dioxide from the enclosed bioreactor and optionally processing the carbon dioxide in a useful manner, for example by providing the same as feedstock for a biomass-producing reactor as described in our U.S. Ser. No. 63/364,275, the contents of which are hereby incorporated by reference, for example.

The method may further comprise harvesting hydrogen from the enclosed bioreactor at a hydrogen harvesting rate, and separating the hydrogen from other gasses by filtering the hydrogen through a hydrogen-selective membrane filter and transferring the hydrogen into a hydrogen storage container.

Forming the synthetic reaction mixture may comprise adding the at least one hydrogen producing microorganism until a percentage of the hydrogen producing microorganism in the synthetic reaction mixture is at least about 10% or at least about 20% of a total amount of microorganisms in the synthetic reaction mixture.

The enclosed bioreactor may have a volume of at least about 100 m3, or at least about 103 m3, or at least about 104 m3, or at least about 105 m3. The enclosed bioreactor may have a volume of up to about 4×109 m3, or up to about 4×108 m3, or up to about 4×107 m3, or up to about 4×106 m3. The enclosed bioreactor may have a volume of from about 100 m3 to about 4×109 m3, or from about 100 m3 to about 4×106 m3, or from about 4×106 m3 to about 4×109 m3.

The hydrogen storage container may be a gas tank, a hydrogen subterranean formation, or a hydrogen artificial enclosure.

The hydrogen subterranean formation may include a natural formation or non-natural formation.

The hydrogen artificial enclosure may be made of one or more building materials. The building materials may include a cement, a plastic, a non-natural rubber, a geomembrane of any kind, concrete, a metal or metal alloy (such as steel), or a combination thereof.

The following examples are offered by way of illustration of certain embodiments of aspects of the application herein. None of the examples should be considered limiting on the scope of the application.

### EXAMPLES

### Example 1: Field Well Trial

Schematically illustrated (for a single well) in FIG. 1, two low producing oil wells was stimulated in a huff-n-puff application to increase microbial hydrogen production. An oil sample from each well was taken and its indigenous microbiological content determined, with the results set out in Tables 1 and 2 below:

TABLE 1
Well 1-Indigenous microbial population:

| | |
|---|---|
| Halanaerobium praevalens DSM 2228 | 18.1% |
| Acinetobacter johnsonii | 13.4% |
| Desulfohalobium retbaense DSM 5692 | 12.1% |
| Halanaerobium hydrogeniformans | 7.4% |
| Methanohalophilus halophilus | 6.0% |
| Methanohalophilus mahii DSM 5219 | 4.7% |
| Escherichia coli | 2.0% |
| Halobacteroides halobius DSM 5150 | 2.0% |
| Azospirillum thiophilum | 1.3% |
| Keratinibaculum paraultunense | 1.3% |

TABLE 2
Well 2-Indigenous microbial population:

| | |
|---|---|
| Methanohalophilus halophilus | 13.2% |
| Methanohalophilus mahii DSM 5219 | 11.0% |
| Halanaerobium praevalens DSM 2228 | 7.3% |
| Desulfohalobium retbaense DSM 5692 | 6.8% |
| Halanaerobium hydrogeniformans | 3.7% |
| Acinetobacter johnsonii | 3.2% |
| Petrotoga mobilis SJ95 | 3.2% |
| Halothermothrix orenii H 168 | 2.3% |
| Flexistipes sinusarabici DSM 4947 | 2.3% |
| Pelobacter acetylenicus | 1.8% |
| Methanotorris igneus Kol 5 | 1.4% |
| Bacillus mycoides | 1.4% |

In the first well, nutrients were blended as described below in Table 3 into 500 bbls of produced water in a frac tank.

TABLE 3
Nutrient package mixed into the 500 bbls:

| Reagent | [g/L] |
|---|---|
| K₂HPO₄ | 1.044 |
| NH₄Cl | 1.5 |
| Sucrose | 1.41 |
| Yeast extract | 1.5 |
| Tween 80 | 0.081 |

The nutrient mix was injected down the annulus of the well and an additional 500 bbls of produced water was pumped down the annulus on top of the nutrient mixture. In the second well, the same process occurred with the exception that a consortium of microbes capable of producing hydrogen from hydrocarbon fermentation is incubated in a transportable microbiological incubator and added to the first 500 bbls of produced water along with the nutrient package.

The consortium is prepared by combining in the transportable microbiological incubator hydrogen producing microorganisms selected to be different from the indigenous microbial populations, and for their capability to digest hydrocarbons to yield hydrogen in preference to methane, in the proportions identified in Table 4:

TABLE 4
Well 2-Exogenous microbial population:

| | |
|---|---|
| Pseudothermotoga elfii | ~20% |
| Pseudothermotoga hypogea | ~20% |
| Thermotoga petrophila | ~20% |
| Petrotoga mobilis | ~20% |
| Caldanaerobacter tengcongensis | ~20% |

The exogenous microbes are maintained in the transportable microbiological incubator in anaerobic liquid culture and nurtured for 2 months under nitrogen (100% N2) at 150 F (65.56 degC), with fresh media inoculated every 3-4 days. The selected media is an ATCC 2114 medium modified for preferential culturing of extremophiles.

Approximately 400L of microbial culture consisting of approximately 10⁸ cells/mL is added to the 500 bbls.

Following addition of the nutrient package (Well 1) and the nutrient/microbial consortium package (Well 2), the two wells are shut-in for 4 days. After the four-day shut-in period the wells are opened and samples are collected off the gas flow line for analysis with respect to H2 content on a gas chromatograph, with the results presented in Table 5 below:

TABLE 5
Gas Chromatography characterization of samples:

| | Baseline H2 | After shut-in |
|---|---|---|
| Well | (ppm) | H2(ppm) |
| 1 (nutrients only) | <112 (LOD) | 1761 |
| 2 (nutrients and microbes) | <112 (LOD) | 13251 |

The gas chromatography is carried out using a standard protocol as follows: 10 milliliter gas samples are extracted from culture bottles using 10 milliliter plastic luer lock syringes. Field gas samples are collected in multi-layer foil gas sampling bags connected via tygon tubing to a sampling valve directly off the of wellhead flow line. Gas samples are injected immediately into the inlet port of an SRI 8610C Gas Chromatograph. The sample is analyzed using a Flame Photometric Detector (FPD), a Flame Ionization Detector (FID), an FID with a large methanizer (FIDM), and a Thermal Conductivity Detector (TCD).

The samples are passed through an 18-inch HayeSep D Packed Column, a 3-foot Molecular Sieve 5A Packed Column, and then into the TCD and FIDM detectors following relay G injection. When relay F is turned on the samples are run through a 6-foot HayeSep D Column and a 60-meter MXT-1 Capillary Column before being analyzed using the FID and FPD. The G relay is turned on at time 0.020 minutes and is turned off at 1.000 minutes, while the F relay is turned on after 4.500 minutes. The initial temperature is set for 50° C. and held for 6 minutes before ramping to 270° C. at a rate of 30° C. per minute. The temperature is held at 270° C. for 6.500 minutes to remove excess sample from the columns.

Any peak areas produced are converted into ppm values using the trend lines of calibration curves derived from standards of various concentrations.

It will be seen from the results in Table 5 that modifying the composition of the well by the introduction into the well of a nutrient package and of consortium of hydrogen producing microorganisms incubated in a transportable microbiological incubator and selected positively to diversify the microbiological abundance of hydrogen-producing microorganisms in the well and for the preferential production of hydrogen over methane increases hydrogen production from the well by two orders of magnitude with respect to baseline H2 production, and by an order of magnitude with respect to introduction of the nutrient package alone.

### Example 2: Microbe Laboratory Data

The consortium of microbes described in Example 1 and capable of producing hydrogen from hydrocarbon fermentation is used to inoculate 6 different synthetic seawater blends in triplicate as described below in Table 6.

TABLE 6
Synthetic seawater blends:

| Brine | Description |
|---|---|
| A | Synthetic seawater |
| B | Synthetic seawater with oil |
| C | Synthetic seawater with nutrients |
| D | Synthetic seawater with nutrients and oil |
| E | Synthetic seawater with enhanced nutrients |
| F | Synthetic seawater with enhanced nutrients and oil |
| G | Synthetic seawater with algae biomass and oil |

Synthetic seawater is a simple reproducible representative of produced water brines. It is produced using NeoMarine aquarium salts by Brightwell Aquatics. The oil used in this example is a sweet west Texas crude blend (API 25-35). 4 mL of the oil is used in 100 mL synthetic seawater sample. The nutrient packages employed are as follows in Tables 7, 8 and 9:

TABLE 7
Synthetic seawater with nutrients:

| Reagent | [g/L] |
|---|---|
| Aquarium Salts | 35.40290621 |
| K₂HPO₄ | 0.348 |
| KH₂PO₄ | 0.227 |
| NH₄Cl | 0.5 |
| Wolfes Vitamin solution | 10 mL |
| Reducing agent | 1 |
| Resazurin solution | ~1 mL |
| dH₂O | 999 mL |
| Combine, pH to desired | |
| 6.5 +− 0.5), filter sterilize | |

TABLE 8
Synthetic seawater with enhanced nutrient package:

| Reagent | [g/L] |
|---|---|
| Aquarium Salts | 35.40290621 |
| K₂HPO₄ | 0.348 |
| NH₄Cl | 0.5 |
| Glucose | 0.47 |
| Yeast extract | 0.5 |
| Tween 80 | 0.027 |
| Reducing agent | 1 |
| Resazurin solution | ~1 mL |
| dH₂O | 999 mL |
| Combine, pH to desired | |
| 6.5 +− 0.5), filter sterilize | |

TABLE 9
Synthetic seawater with algae biomass nutrient package:

| Reagent | [g/L] |
|---|---|
| Aquarium Salts | 35.40290621 |
| K₂HPO₄ | 0 |
| NH₄Cl | 0 |
| Glucose | 0 |
| Yeast extract | 0 |
| Chlorella algae powder | 0.5 |
| Tween 80 | 0.027 |
| Reducing agent | 1 |
| Resazurin solution | ~1 mL |
| dH₂O | 999 mL |

A 100 mL sample of each brine A-E is prepared anaerobically in glass bottles and sealed. Following inoculation, the bottles are incubated at 65 C for 48 hours along with abiotic controls for each brine.

At 48 hours, samples are taken for ATP analysis (microbial enumeration) and gas analysis, the results of which are shown in Table 10.

TABLE 10
ATP Analysis:

| | | Abiotic Control | | Inoculated | |
|---|---|---|---|---|---|
| | | H2 | Microbial | H2 | Microbial |
| | | Concen- | enumera- | Concen- | enumera- |
| | | tration | tion | tration | tion |
| Brine | Description | (ppm) | (cells/mL) | (ppm) | (cells/mL) |
| A | Synthetic seawater | 0 | 6.92E+03 | 0 | 1.51E+06 |
| B | Synthetic seawater | 0 | 2.00E+04 | 82.33 | 7.54E+06 |
| | with oil | | | | |
| C | Synthetic seawater | 0 | 1.40E+03 | 119 | 7.55E+06 |
| | with nutrients | | | | |
| D | Synthetic seawater | 0 | 7.67E+03 | 1406.7 | 2.58E+07 |
| | with nutrients | | | | |
| | and oil | | | | |
| E | Synthetic seawater | 0 | 9.80E+03 | 62.33 | 3.04E+07 |
| | with enhanced | | | | |
| | nutrients | | | | |
| F | Synthetic seawater | 0 | 2.95E+04 | 2735.33 | 2.07E+07 |
| | with enhanced | | | | |
| | nutrients and oil | | | | |
| G | Synthetic sea water | 0 | 1.18E+07 | 2365.5 | 1.01E+08 |
| | with algae biomass | | | | |
| | and oil | | | | |

In sample E, the enhanced nutrient package used causes rapid microbial growth at 24 hours and all of the carbon source is consumed which leads to a lower reading at 48 hours when no oil is present to maintain microbial activity, which rationalizes the lower H₂ concentration observed for this sample relative to comparative sample C.

The test kit used for the determination of ATP is the Luminultra QGO-M which is compliant with ASTM Standard E2694 for the measurement of ATP in Metalworking Fluids and D7687 for the measurement of ATP in fuels, fuel/water mixtures and fuel-associated water.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary.

1. A process for the microbial production of hydrogen from the site of hydrocarbonaceous deposit, the process comprising:
modifying the composition of the deposit through the introduction into or into the vicinity of the deposit of at least one hydrogen producing microorganism,
wherein the at least one hydrogen producing microorganism is provided on site by means of a transportable microbiological incubator.

2. A process according to claim 1, wherein the at least one hydrogen producing microorganism comprises a non-native microorganism.

3. A process according to claim 1, wherein the incubator is containerized in operationally intact form.

4. A process according to claim 1, wherein the incubator is provided with means for nurturing the at least one hydrogen producing microorganism.

5. A process according to claim 4, wherein the means for nurturing comprise means for providing the at least one hydrogen producing microorganism in the incubator with one or more nutrients and/or adjuvants for encouraging or selectively encouraging the growth of the at least one hydrogen producing microorganism in the incubator.

6. A process according to claim 4, wherein the means for nurturing comprise means for maintaining conditions of temperature, pressure and/or chemical environment within the incubator conducive to the incubation of the at least one microorganism.

7. A process according to claim 1, wherein the hydrogen producing microorganism contained in the transportable microbiological incubator is selected positively to diversify the microbial abundance of hydrogen-producing microorganisms in the deposit when charged thereto.

8. A process according to claim 1, wherein the hydrogen producing microorganism contained in the transportable microbiological incubator is selected for the preferential production of hydrogen over methane.

9. The process according to claim 1, wherein the at least one hydrogen producing microorganism is:
a. a microorganism not naturally present in the hydrocarbonaceous deposit; and/or
b. of a strain of microorganisms not naturally present in the hydrocarbonaceous deposit; and/or
c. of a species of microorganisms not naturally present in the hydrocarbonaceous deposit; and/or
d. of a genus of microorganisms not naturally present in the hydrocarbonaceous deposit;
e. a microorganism naturally present in the hydrocarbonaceous deposit; and/or
f. a microorganism naturally present in the hydrocarbonaceous deposit but genetically modified to increase (relative to the naturally present microorganism) its propensity for hydrogen production by the metabolization by that microorganism of one or more hydrocarbons contained within the deposit.

10. The process according to claim 1, wherein the at least one hydrogen producing microorganism is one of a plurality of different hydrogen producing microorganisms, strains of microorganisms, species of microorganisms, genera of microorganisms and/or naturally occurring, optionally genetically modified, organisms introduced into the deposit.

11. The process according to claim 10, wherein the plurality is greater than two, greater than three, greater then four, greater than five and/or greater than ten.

12. The process according to claim 11, wherein the at least one hydrogen producing microorganism is introduced into the deposit from the transportable microbiological incubator and accompanied during, after or upon its introduction by at least one nutrient selected to promote the growth of said microorganism and introduced into the deposit for that purpose, optionally wherein the at least one nutrient is are supplied from a nutrient reservoir, optionally a nutrient reservoir contained within the incubator.

13. The process according to claim 1, wherein the at least one hydrogen producing microorganism is introduced into the deposit and accompanied during, after or upon its introduction by at least one pH regulator selected to regulate the pH environment in which the microorganism resides in the deposit and introduced into the deposit for that purpose.

14. The process according to claim 13, wherein the pH regulator is selected to regulate the pH of the hydrogen producing microorganism environment in the deposit to a pH within the range of from about 5 to about 9, from about 6 to about 8 and/or from about 6 to about 7.

15. The process according to claim 13, wherein the pH regulator also serves as a nutrient.

16. The process according to claim 1, wherein the hydrogen producing microorganism is introduced into the deposit and accompanied during, after or upon its introduction by at least one reducing agent.

17. The process according to claim 1, wherein the hydrocarbonaceous deposit is a liquid hydrocarbonaceous deposit.

18. The process according to claim 1, wherein the at least one hydrogen producing microorganism has a genus of Syntrophobacter, Syntrophus, Syntrophomonas, Thermoanaerobacter, Thermotoga, Pseudothermotoga, Thermoanaerobacterium, Fervidobacterium, Thermosipho, Haloanaerobium, Acetoanaerobium, Anaerobaculum, Geotoga, Petrotoga, Thermococcus, Pyrococcus, Clostridium, Enterobacter, Klebsiella, Ethanoligenens, Pantoea, Escherichia, Bacillus, Caldicellulosiruptor, Pelobacter, Caldanaerobacter, Marinitoga, Oceanotoga, Defluviitoga, Kosmotoga, Caloranaerobacter or a combination or mixture thereof.

19. The process according to claim 1, wherein conditions within the incubator at least to some extent replicate one or more conditions (of temperature, pressure and/or chemical environment) of the site of hydrocarbonaceous deposit.

20. Plant for the microbial production of hydrogen from a hydrocarbonaceous deposit comprising:
means in the form of a transportable microbiological incubator for generating at least one hydrogen producing microorganism; and
means for supplying the at least one at least one hydrogen producing microorganism into the hydrocarbonaceous deposit.

21. Plant according to claim 20, wherein additionally comprising means for extracting from the hydrocarbonaceous deposit a product stream comprising at least hydrogen gas generated by the microbiological action in the deposit of the at least one hydrogen producing microorganism.

22. Plant according to claim 21, wherein the product stream comprises useful product streams besides hydrogen—optionally oil and/or methane.

23. Plant according to claim 22, wherein the product stream further comprises an aqueous phase deriving at least in part from an aqueous carrier for the at least one hydrogen producing microorganism supplied to the hydrocarbonaceous deposit.

24. Plant according to claim 22, wherein comprising means for separating product stream phases and for recycling at least part of any aqueous phase to the deposit, optionally with the incorporation of fresh microbiological material from the incubator.

25. Plant according to claim 24, wherein comprising a three phase separator effective to isolate independently gaseous, oleaginous and aqueous streams from the product stream.
